(19) United States
(12) Patent Application Publication
(10) Pub. No.: US 2020/0171240 A1
(43) Pub. Date: Jun. 4, 2020

(54) Method for Operating an Implantable, Gas Pressure-Operated Infusion Pump Comprising a Throttle Portion

(71) Applicant: tricumed Medizintechnik GmbH, Kiel (DE)

(72) Inventor: Karl-Heinz OTTO, Kiel (DE)

(21) Appl. No.: 16/373,790

(22) Filed: Apr. 3, 2019

(30) Foreign Application Priority Data
Dec. 3, 2018 (DE) 10 2018 130 655.5

(51) Int. Cl. A61M 5/168; A61M 5/155

(52) U.S. Cl. CPC A61M 5/16881; A61M 5/155; A61M 2205/3334; A61M 2205/52; A61M 2205/3569; A61M 2205/3389

(57) ABSTRACT

An implantable, gas pressure-operated infusion pump includes a throttle portion, a controllable valve upstream or downstream of the throttle portion, and a controller that controls the opening duration and/or opening frequency of the valve. A method for operating the infusion pump includes determining a standard volume of a standard infusate dispensed by the infusion pump within a pre-determined time period and determining an infusate volume of an infusate, having a different composition than the standard infusate, dispensed by the infusion pump within the pre-determined time period. The method further includes operating the infusion pump together with the infusate at an opening duration and/or opening frequency of the valve that is reduced or increased compared with operating the infusion pump together with the standard infusate in order to dispense a volume of infusate substantially the same as the standard volume.

### CROSS REFERENCE TO RELATED APPLICATIONS

This application claims the benefit of German Patent Application No. 10 2018 130 655.5, titled “Method for Operating an Implantable, Gas Pressure-Operated Infusion Pump Comprising a Throttle Portion” and filed on Dec. 3, 2018, which is incorporated herein by reference.

### TECHNICAL FIELD

The invention relates to a method for operating an implantable, gas pressure-operated infusion pump comprising a throttle portion, a controllable valve upstream or downstream of the throttle portion, and a controller that controls the opening duration and/or opening frequency of the valve.

### BACKGROUND

An infusion pump of this kind is already known from European Patent Application Publication No. EP 3 247 422 A1, which is incorporated herein by reference. The known infusion pump is designed to compensate for infusate pressure fluctuations resulting from temperature changes in the propellant and/or changes in the spring rate caused by the filling level of the infusate reservoir. The pump changes the opening duration and/or opening frequency of the valve to compensate for the infusate pressure functions.

The advantage of controllable, gas pressure-operated infusion pumps compared with gas pressure-operated infusion pumps having a constant flow rate is that the drug dose can be set, or the drug dose can be adjusted during treatment, by a control device, without it being necessary to change the entire infusate. Rather, a daily dose adjusted for the patient can be achieved by changing the opening duration and/or opening frequency of the valve while keeping the drug concentration of the infusate the same.

Even if the known infusion pumps are, in principle, indispensable for treating particular diseases, using different infusates also leads to varying degrees of therapeutic success. The varying degrees of success are not caused by the pharmaceutical effect of the infusates but instead from the combination of the infusate and the infusion pump.

The object of the invention is therefore that of providing an infusion pump that can be used equally successfully for different infusates.

### SUMMARY OF THE INVENTION

An implantable, gas pressure-operated infusion pump comprises a throttle portion, a controllable valve upstream or downstream of the throttle portion, and a controller that controls the opening duration and/or opening frequency of the valve. The pump is configured to dispense a drug contained in an infusate. The controller is designed to dispense a pre-defined drug dose within a pre-determined time period by reducing or increasing the opening duration and/or opening frequency of the valve based on the viscosity of the infusate in order to compensate for the viscosity of the infusate differing from the viscosity of a standard infusate.

A memory is connected to the controller and is intended for storing a plurality of reduced or increased opening durations and/or opening frequencies that are assigned to infusates. The memory does not necessarily need to be a component of the infusion pump itself. Instead, the memory can be provided in or on an external control device that wirelessly interconnects the memory and the controller. In this case, the values can be stored in the form of a table, such that the user simply has to select the corresponding drug in each case, and the reduced or increased opening durations and/or opening frequencies, at a specified concentration of the drug in the infusate and a specified dose, are automatically provided to the controller.

A method for operating the infusion pump includes determining a standard volume of a standard infusate dispensed by the infusion pump within a pre-determined time period when the infusion pump is operated at a specified first opening duration and/or a specified first opening frequency of the valve. The method also includes determining the volume of an infusate, having a different composition than the standard infusate, dispensed by the infusion pump within the pre-determined time period when the infusion pump is operated at the specified first opening duration and/or the specified first opening frequency of the valve. The method then includes calculating the difference between the volume of the standard infusate dispensed by the infusion pump and the volume of the infusate dispensed by the infusion pump. For the infusate, the infusion pump is operated at a second opening duration and/or second opening frequency of the valve, which is reduced or increased compared with operating the infusion pump with the standard infusate, in order to dispense an infusate volume substantially the same as the standard volume.

The preceding summary is provided to facilitate an understanding of some of the innovative features unique to the present disclosure and is not intended to be a full description. A full appreciation of the disclosure can be gained by taking the entire specification, claims, drawings, and abstract as a whole.

### BRIEF DESCRIPTION OF THE DRAWINGS

The disclosure may be more completely understood in consideration of the following description of various illustrative embodiments in connection with the accompanying drawings.

FIG. 1 is a schematic view of an infusion pump in accordance with a first preferred embodiment of the invention.

FIG. 2 is a schematic view of an infusion pump in accordance with a second preferred embodiment of the invention.

FIG. 3 is a schematic view of an infusion pump in accordance with a third preferred embodiment of the invention.

FIG. 4 is a schematic view of an infusion pump in accordance with a fourth preferred embodiment of the invention.

FIG. 5 is a flowchart of a method of operating an implantable, gas pressure-operated infusion pump in accordance with the invention.

### DETAILED DESCRIPTION OF THE INVENTION

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment can be incorporated into an additional embodiment unless clearly stated to the contrary. While the disclosure is amenable to various modifications and alternative forms, specifics thereof have been shown by way of example in the drawings and will be described in detail. It should be understood, however, that the intention is not to limit aspects of the disclosure to the particular illustrative embodiments described. On the contrary, the intention is to cover all modifications, equivalents, and alternatives falling within the spirit and scope of the disclosure.

As used in this specification and the appended claims, the singular forms “a”, “an” and “the” include plural referents unless the content clearly dictates otherwise. As used in this specification and the appended claims, the term “or” is generally employed in its sense including “and/or” unless the content clearly dictates otherwise.

In the description of embodiments disclosed herein, any reference to direction or orientation is merely intended for convenience of description and is not intended in any way to limit the scope of the present invention. Relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise.

In the present case, it has been found that the therapeutic success of an infusate depends not only on the infusate used, alone, or on the infusion pump used, alone, but rather on the combination of the infusate used and the infusion pump used. It has been found, in particular, that the varying viscosity of different infusates results in different conveyed volumes of infusate at an identical opening duration and/or opening frequency of the valve of a controllable infusion pump. The basic concept of the invention is to account for the viscosity of an infusate, when programming the controllable infusion pump, by adjusting the opening duration and/or the opening frequency of the valve.

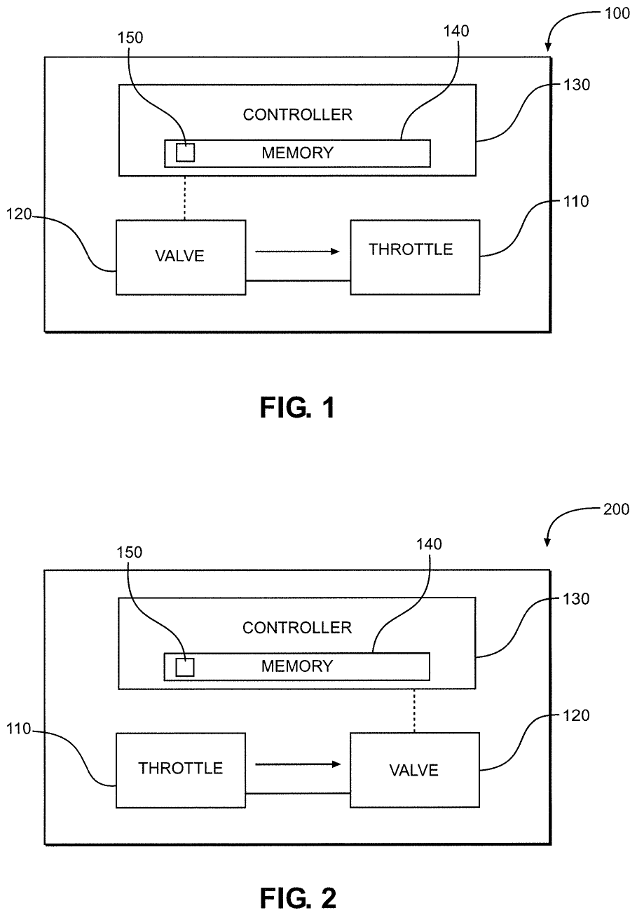

Referring now to FIG. 1, there is shown an implantable, gas pressure-operated infusion pump 100 for dispensing a drug contained in an infusate. Pump 100 comprises a throttle portion 110 and a controllable valve 120 upstream or downstream of throttle portion 110. The upstream arrangement is shown in FIG. 1, while a downstream arrangement is shown as pump 200 in FIG. 2. A controller 130 controls the opening duration and/or opening frequency of valve 120. Controller 130 is configured to dispense a pre-defined drug dose within a pre-determined time period by reducing or increasing the opening duration and/or opening frequency of valve 120 based on a viscosity of the infusate in order to compensate for the viscosity of the infusate differing from a viscosity of a standard infusate.

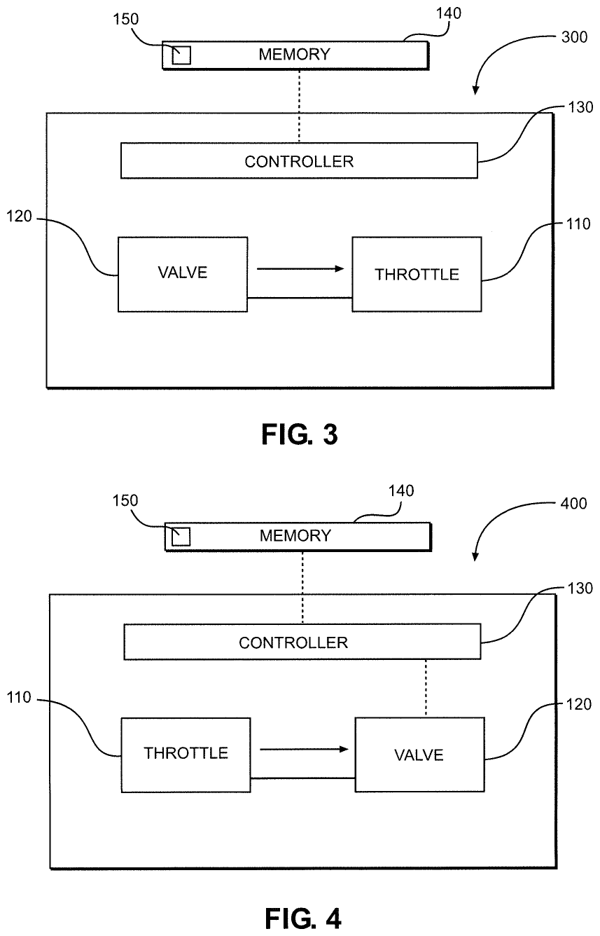

A memory 140 is connected to controller 130 and is configured to store a list/table 150 of a plurality of reduced or increased opening durations and/or opening frequencies that are assigned to infusates. Memory 140 does not necessarily need to be a component of infusion pump 100 itself. Instead, memory 140 can be provided in or on an external control device that wirelessly interconnects memory 140 and controller 130, in pumps 300 and 400 as best seen in FIGS. 3 and 4. In this case, the values can be stored in the form of table 150, such that the user simply has to select the corresponding drug in each case, and the reduced or increased opening durations and/or opening frequencies, at a specified concentration of the drug in the infusate and a specified dose, are automatically provided to controller 130. More details of an infusion pump can be found in U.S. patent application Ser. No. 16/299,562, titled “Pressurized-Gas-Driven Infusion Pump and Method of Operating Such an Infusion Pump” and filed on Mar. 12, 2019, which is incorporated herein by reference.

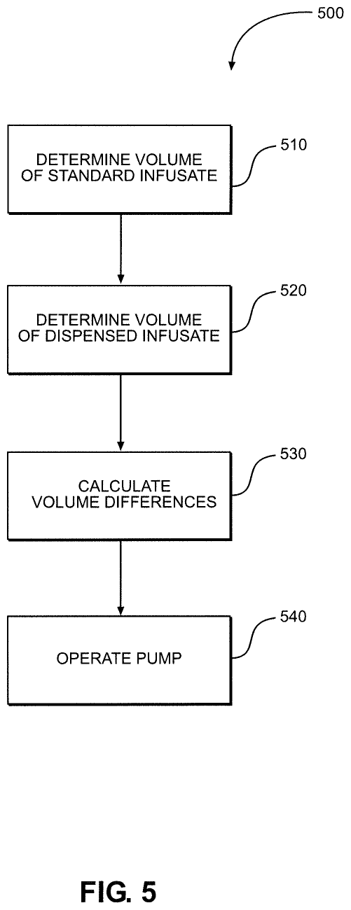

FIG. 5 shows a method 500 for operating implantable, gas pressure-operated infusion pump 100. A step 510 involves determining a standard volume of a standard infusate dispensed by infusion pump 100 within a pre-determined time period when infusion pump 100 is operated at a specified first opening duration and/or a specified first opening frequency of valve 120. A step 520 involves determining an infusate volume of an infusate, having a different composition than the standard infusate, dispensed by infusion pump 100 within the pre-determined time period when infusion pump 100 is operated at the specified first opening duration and/or the specified first opening frequency of valve 120. A step 530 involves calculating the difference between the standard volume and the infusate volume dispensed by infusion pump 100. A step 540 involves, for the infusate, operating infusion pump 100 at a second opening duration and/or second opening frequency of valve 120, which is reduced or increased compared with operating infusion pump 100 together with the standard infusate, in order to dispense a volume of the infusate identical, or substantially equal, to the standard volume.

Preferably, the pre-determined time period for determining the volumes is longer than the first opening duration. More preferably, the pre-determined time period for determining the volumes is more than twice the length of the first opening duration.

The standard infusate is preferably a saline solution, in particular, an aqueous isotonic saline solution (0.9% NaCl). However, owing to the relative measurement method, other solutions can be used as the standard infusate, in particular infusates already used in treatment. Preferably, the infusate contains a drug.

The present invention allows for safe and extremely precise operation of a controllable implantable infusion pump. This is advantageous, in particular, when dispensing high-titer and/or high-strength drugs because the therapeutic success can be ensured. The invention also ensures the therapeutic success of infusates that comprise not only substances that are dissolved or dispersed in the infusate but also contain other structures, such as exosomes or live or dead cells.

Having thus described several illustrative embodiments of the present disclosure, those of skill in the art will readily appreciate that yet other embodiments can be made and used within the scope of the claims hereto attached. Numerous advantages of the disclosure covered by this document have been set forth in the foregoing description. It will be understood, however, that this disclosure is, in many respects, only illustrative. Changes can be made in the details. The disclosure's scope is, of course, defined in the language in which the appended claims are expressed.

1. A method for operating an implantable, gas pressure-operated infusion pump including a throttle portion, a controllable valve upstream or downstream of the throttle portion, and a controller that controls the opening duration or opening frequency of the controllable valve, the method comprising:
a) determining a standard volume of a standard infusate dispensed by the implantable, gas pressure-operated infusion pump within a pre-determined time period when the implantable, gas pressure-operated infusion pump is operated at a specified first opening duration or a specified first opening frequency of the controllable valve;
b) determining an infusate volume of an infusate, having a different composition than the standard infusate, dispensed by the implantable, gas pressure-operated infusion pump within the pre-determined time period when the implantable, gas pressure-operated infusion pump is operated at the specified first opening duration or the specified first opening frequency of the controllable valve;
c) calculating the difference between the standard volume and the infusate volume dispensed by the implantable, gas pressure-operated infusion pump; and
d) for the infusate, operating the implantable, gas pressure-operated infusion pump at a second opening duration or second opening frequency of the controllable valve, which is reduced or increased compared with operating the implantable, gas pressure-operated infusion pump together with the standard infusate, in order to dispense a volume of the infusate substantially equal to the standard volume.

2. The method according to claim 1, wherein the pre-determined time period is longer than the specified first opening duration.

3. The method according to claim 2, wherein the pre-determined time period is more than twice the length of the specified first opening duration.

4. The method according to claim 1, wherein the standard infusate is a saline solution.

5. The method according to claim 1, wherein the infusate contains a drug.

6. An implantable, gas pressure-operated infusion pump for dispensing a drug contained in an infusate, the pump comprising:
a throttle portion;
a controllable valve upstream or downstream of the throttle portion; and
a controller that controls an opening duration or opening frequency of the controllable valve, wherein the controller is configured to dispense a pre-defined drug dose within a pre-determined time period by reducing or increasing the opening duration or opening frequency of the controllable valve based on a viscosity of the infusate in order to compensate for the viscosity of the infusate differing from a viscosity of a standard infusate.

7. The implantable, gas pressure-operated infusion pump according to claim 6, further comprising a memory that is connected to the controller and stores a plurality of reduced or increased opening durations or opening frequencies that are assigned to infusates.

8. The implantable, gas pressure-operated infusion pump according to claim 7, wherein the memory and the controller are wirelessly interconnected.
